(19) United States
(12) Patent Application Publication
(10) Pub. No.: US 2009/0211572 A1
(43) Pub. Date: Aug. 27, 2009

(54) ENDOTRACHEAL INTUBATION APPARATUS PROVIDING ENHANCED STABILITY IN AN INTUBATED PATIENT

(76) Inventor: Paul A. Matera, Annapolis, MD (US)

Correspondence Address: WOMBLE CARLYLE SANDRIDGE & RICE, PLLC, ATTN: PATENT DOCKETING, P.O. BOX 7037, ATLANTA, GA, 30357-0037, US

(21) Appl. No.: 11/719,623

(22) PCT Filed: Nov. 18, 2005
(86) PCT No.: PCT/US05/42026

§ 371 Date: Apr. 30, 2009

Related U.S. Application Data
(60) Provisional application No. 60/628,941, filed on Nov. 19, 2004.

(51) Int. Cl. A61M 16/04

(52) U.S. Cl. 128/200.26

(57) ABSTRACT

An endotracheal intubation apparatus includes a tube having a distal end, a proximal end, and an extensible and contractible portion. The apparatus further includes at least one cuff on the tube proximate to the distal end. The cuff is movably attached to the tube such that the cuff is capable of at least some movement in a proximal direction and in a distal direction on the tube. A tube holder includes a resilient support configured to substantially bias the proximal end of the tube toward a selected oral position in a patient. The apparatus also includes at least one tube locator that is adapted to indicate the position of a distal portion of the tube within the patient.

### RELATED APPLICATIONS

This application claims priority from U.S. application Ser. No. 60/628,941, filed Nov. 19, 2004, which is hereby incorporated by reference in its entirety.

### FIELD OF THE INVENTION

The invention relates to medical devices for artificially ventilating patients, and more particularly relates to an endotracheal intubation apparatus that accommodates bodily movement of an intubated patient, and at least some displacement of a proximal portion of the device, such that the tracheal position of distal portions of the apparatus in the patient remains substantially unaltered.

### BACKGROUND

Human medical patients sometimes require artificial ventilation during surgery or medical emergencies. For example, accident victims sometimes require coronary pulmonary resuscitation (CPR), or endotracheal intubation by an emergency medical technician, or by an operating room anesthesiologist. Often, medical personnel establish a ventilation passageway by inserting an elongated tube through a patient's nose or mouth, and into the patient's trachea. Such a tube commonly is known as an endotracheal tube, or “ETT.” Other surgical procedures also sometimes require use of an ETT to collapse one lung, such as to take a biopsy from the lung, to repair a lobar defect, to remove a tumor, to repair an abscess, or to perform esophageal triage. Generally, ETT's used to intentionally collapse a lung include two separate air passageways or “lumens.”

Endotracheal intubation typically is accomplished by inserting an ETT through the patient's mouth or nasal passages and into the airway passage or trachea. Known ETT's generally include a substantially pliable elongated tube having one end configured for connection to a respirator or other air supply mechanism for introduction of air into a patient's lungs. Such tubes commonly are constructed of medical grade plastic or rubber, such as medical grade poly vinyl chloride (PVC). ETT's typically range in size from about 3 to about 9 millimeters in internal diameter. Medical personnel select an ETT that corresponds in size to a patient's body size. ETT's typically include a “distal” or insertion end (that end of the tube which is inserted into a patient), and a “proximal” end (that end of the tube that outwardly extends from the patient, and is connectable to an air supply device). Air is supplied through the tube to a patient's trachea and lungs.

In order to insert an ETT, an operator typically places a laryngoscope in the mouth of the patient. The blade portion of the laryngoscope is used to push the tongue laterally, as the operator applies a lifting force to the handle of the laryngoscope in order to visualize the anatomical structures of the mouth and airway. The target insertion point of the ETT is the glottis, which is the opening between the vocal cords and the inlet to the trachea. The distal end of the ETT is inserted into the glottis.

In some patients, placing the insertion end of the ETT through the glottis can be difficult. In such cases, an operator typically must remove the laryngoscope, insert a metal or plastic stylet (semi-rigid wire) into the ETT, and bend the tube and stylet into an appropriate configuration to aid in placing and guiding the insertion tip. A small bend, resembling the shape of a hockey stick, commonly is made in the stylet and the distal end of the ETT. The bent shape of the insertion tip facilitates passing the tip through the glottis and into the trachea.

Many ETT's include at least one inflatable balloon cuff on a distal portion of the tube. As used herein, the term “distal” or “distal portion” refers to a portion of an ETT that is nearer to the distal end of the ETT than to the proximal end. In addition, the term “distal direction” is used herein to refer to a direction that is substantially toward the distal end of the ETT. Conversely, the term “proximal” or “proximal portion” is used herein to refer to a portion of an ETT that is nearer to the proximal end of the ETT than to the distal end. In addition, the term “proximal direction” is used herein to refer to a direction that is substantially toward the proximal end of the ETT. A balloon cuff conventionally is located in a position along the ETT that causes the cuff to engage the inner wall of the trachea (or pharynx or larynx, depending upon the specific design of the ETT). When the ETT is in place, the cuff is inflated such that the cuff forms a substantially airtight seal between the tube and the surrounding body tissue, thereby preventing the escape of air delivered through the ETT to the lungs.

Typically, balloon cuffs are thin-walled bladders that surround a distal portion of the ETT, and are inflated to form a round or ovoid seal that contacts the surrounding tissue or mucosa. Balloon cuffs commonly are inflated by detachable syringes that are connected to small-diameter tubes that extend from the proximal end of the ETT to the cuff. The seals formed by the inflated cuffs preclude the air that has been forced into the patient's lungs from bypassing the lungs and escaping through the patient's trachea or bronchus. Additionally, the seals formed by the inflated cuffs provide a barrier to the flow of blood, mucus, and secretions.

Monitoring the position of the ETT typically is accomplished by noting the depth of insertion of the ETT into a patient. ETT's may include graduated markings along their lengths for this purpose. Medical personnel commonly note or mark the depth of insertion of an ETT that coincides with the patient's teeth, for example. Unfortunately, noting this marking alone may not be sufficient to confirm that the balloon cuff and the distal end of an ETT are in a proper position in a patient.

Conventional designs of endotracheal tubes have several shortcomings. Once placed in an intubated patient, a conventional ETT sometimes can migrate deeper into the trachea, can be accidentally pulled from the trachea, or can otherwise slip from a selected position in a patient. In particular, movement of an intubated patient's head or neck can displace the ETT from its initial selected position in the patient. In addition, the localized pressure exerted by conventional round or ovoid balloon cuffs on surrounding tissue may induce pressure necrosis or vascular compromise in a patient.

Accordingly, there is a need for a dynamically stable endotracheal intubation apparatus that minimizes unwanted migration and displacement of the apparatus once the apparatus has been placed at a selected position within a patient's airway. In addition, there is a need for an endotracheal tube having an expandable cuff that minimizes the degree of localized pressure exerted on surrounding tissues.

### SUMMARY OF THE INVENTION

The invention includes an endotracheal intubation apparatus that includes a tube comprising a distal end, a proximal end, and an extensible and contractible portion. The apparatus also may include at least one expandable cuff on the tube proximate to the distal end, the cuff being movably attached to the tube such that the cuff is capable of at least some movement in a proximal direction and in a distal direction relative to the tube. The apparatus may further include a tube holder comprising a resilient tube support configured to substantially bias the proximal end of the tube toward a central oral position of a patient. In addition, the apparatus may include at least one tube locator adapted to indicate the position of the tube within the patient. The extensible and contractible portion of the tube, the movably attached expandable cuff, and the tube holder with resilient tube support cooperate to prevent migration or slippage of the apparatus once the tube has been inserted into a patient's trachea. The tube locator permits verification that the position of the tube in the patient's trachea is unchanged.

The invention also includes an endotracheal tube having a distal end, a proximal end, and an extensible and contractible portion. The extensible and contractible portion of the tube may include at least one corrugated tube section. In addition, the endotracheal tube may include a distal tube portion and a proximal tube portion that are telescopically interconnected to form the extensible and contractible portion. A sleeve may be provided over the extensible and contractible portion. The sleeve may include at least some corrugations. A protective sheath may be provided over the sleeve.

The invention also includes an endotracheal tube having a distal end and at least one cuff on the tube proximate to the distal end. The cuff is movably attached to the tube such that the cuff is capable of at least some movement in a proximal direction and in a distal direction on the tube. The cuff may be an inflatable balloon cuff, or alternatively, may include a resilient compressible material. The cuff may be movably attached to the tube by at least one flexible membrane.

The invention further includes a tube holder for an endotracheal tube that includes a resilient tube support configured to substantially bias the proximal end of the tube toward a central oral position of a patient. The tube holder may include an anchor that is removably mountable on the patient proximate to the patient's mouth. The resilient tube support may include a resilient arm having a first end connected to the anchor, and a second end configured to removably receive the proximal end of the tube. The resilient arm may be configured to permit at least some movement of the second end and the proximal end of the tube received therein in substantially any direction. An adjustable strap may be provided for removably mounting the anchor on the patient.

The invention also includes an endotracheal tube having a tube locator for use in determining the location of a distal portion of the tube in an intubated patient. The tube locator may include at least one light-emitting member such as a fiber optic element, or may include at least one magnetic member.

These and other aspects of the invention will be understood from a reading of the following detailed description together with the drawings.

### BRIEF DESCRIPTION OF THE DRAWINGS

FIG. 1 is a perspective view of an endotracheal intubation apparatus according to the invention applied to a patient;

FIG. 2A is an elevation view of one embodiment of a tube portion of the endotracheal intubation apparatus of FIG. 1 showing the tube in a contracted state;

FIG. 2B is an elevation view of a tube portion of the endotracheal intubation apparatus of FIGS. 1 and 2A showing the tube in an extended state;

FIG. 3A is a longitudinal cross-sectional view of another embodiment of a tube portion of the endotracheal intubation apparatus of FIG. 1 showing the tube in a contracted state;

FIG. 3B is a longitudinal cross-sectional view of the tube portion of FIG. 3A showing the tube in an extended state;

FIG. 4A is a longitudinal cross-sectional view of a further embodiment of a tube portion of the endotracheal intubation apparatus of FIG. 1 showing the tube in a contracted state;

FIG. 4B is a longitudinal cross-sectional view of the tube portion of FIG. 4A showing the tube in an extended state;

FIG. 5A is a longitudinal cross-sectional view of one embodiment of an inflatable balloon cuff portion of the endotracheal intubation apparatus of FIG. 1, showing the cuff in a deflated state;

FIG. 5B is a longitudinal cross-sectional view of the inflatable balloon cuff of FIG. 5A, showing the cuff in an inflated state;

FIG. 6A is a longitudinal cross-sectional view of the inflated balloon cuff of FIG. 5B;

FIG. 6B is a longitudinal cross-sectional view of the inflated balloon cuff of FIGS. 5B and 6A, showing the tube displaced in a distal direction;

FIG. 6C is a longitudinal cross-sectional view of the inflated balloon cuff of FIGS. 5B, 6A and 6B, showing the tube displaced in a proximal direction;

FIG. 7A is a longitudinal cross-sectional view of another embodiment of an expandable cuff for use in the endotracheal intubation apparatus of FIG. 1, showing the cuff in a compressed state;

FIGS. 7B-7D are longitudinal cross-sectional views of the expandable balloon cuff of FIG. 7A, showing the cuff in progressive stages of expansion;

FIG. 8 is perspective view of a tube holder portion of the apparatus shown in FIG. 1; and

FIGS. 9A-9B are perspective views of a tube holder portion with locking device of the endotracheal intubation apparatus of FIG. 1.

### DETAILED DESCRIPTION

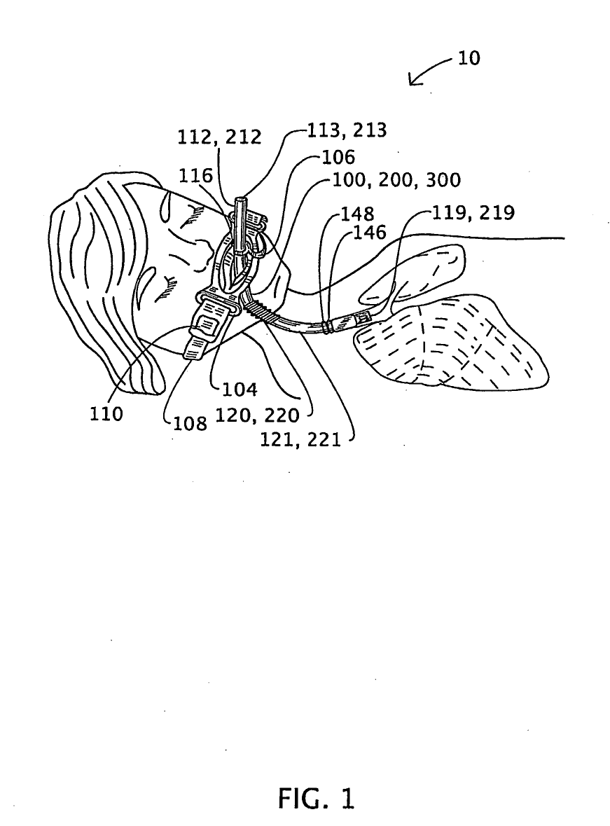

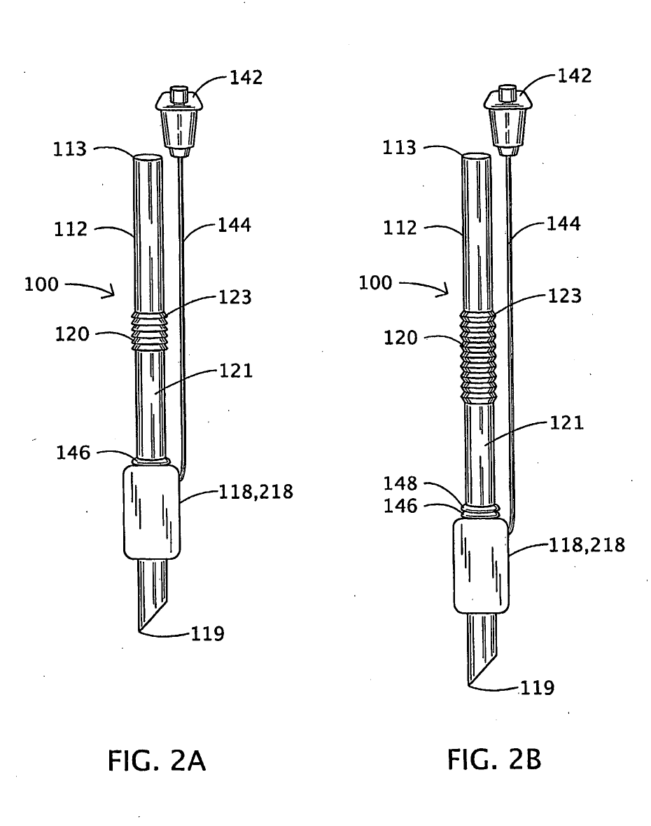

One embodiment of an endotracheal intubation apparatus 10 according to the invention is shown in FIG. 1. In this embodiment, the apparatus 10 includes an endotracheal tube (ETT) 100, 200, 300 and a tube holder 102. As shown in FIG. 1, the ETT 100, 200, 300 includes a proximal end 113, 213 and a distal end 119, 219. The distal end 119, 219 of the ETT 100, 200, 300 may be tapered as shown in FIGS. 2A-2B, may have a square end, or may have any other suitable shape. As shown in FIG. 1, a distal portion 121, 221 of the ETT 100, 200, 300 can be inserted through the mouth of a patient and into the patient's trachea in the usual manner. When so inserted, the proximal end 113, 213 and part of the proximal portion 112, 212 of the ETT outwardly extend from the patient's mouth. The proximal end 113, 213 of the ETT 100, 200, 300 is configured for attachment to a suitable air supply device, such as a ventilator, a valved air bag, or the like, in a manner known in the art. An inflatable or expandable cuff 118, 170, 218 can be provided on the distal portion 121, 221 of the ETT 100, 200, 300 to substantially seal the region between the patient's airway and the distal portion 121, 221 of the ETT. The ETT 100, 200, 300 also can include an extensible and contractible portion 120, 220. The extensible and contractible portion 120, 220 (also referred to herein as the “dynamic buffer”) is capable of being longitudinally extended and longitudinally contracted in response to patient movement, displacement of the proximal end 113 of the ETT 100, or axial loads applied to the ETT 100, 200, 300, thereby minimizing any detrimental effect on the position of the distal portion 121, 221 of the ETT 100, 200, 300 in the intubated patient.

When the extensible and contractible portion 120, 220 is fully compressed, the distance from the cuff 118, 170, 218 to the proximal end 113, 213 of the ETT 100, 200, 300 is at a minimum. In other words, in the fully compressed state of the embodiment shown in FIG. 2A, the proximal end 113 of the ETT 100 is nearest the cuff 118, 218 and the distal end 119. Conversely, when the extensible and contractible portion 120 is fully extended, the distance from the cuff 118, 218 to the proximal end 113 of the ETT 100 is at a maximum. In other words, in the fully extended state of the embodiment shown in FIG. 2B, the proximal end 113 of the ETT 100 is farthest from the cuff 118, 218 and the distal end 119. Additional details of the ETT 100, 200, 300 shown in FIG. 1 are described below.

The tube holder 102 shown in FIG. 1 is configured to substantially maintain the ETT 100, 200, 300 in a preferred position in an intubated patient. For example, the tube holder 102 may be configured to substantially maintain the proximal end 113, 213 and proximal portion 112, 212 of the ETT 100, 200, 300 at a central or other oral position in the patient, and to substantially prevent movement or slippage of the ETT 100, 200, 300 in the intubated patient once the ETT 100, 200, 300 has been properly positioned in the patient. In the embodiment shown in FIG. 1, the tube holder 102 includes an anchor 104 and a tube support 106. The anchor 104 is secured to the patient proximate to the patient's mouth. Preferably, the anchor 104 is shaped to substantially conform to the contours of a patient's face, and to surround the patient's mouth. In the embodiment shown in FIG. 1, the anchor 104 is secured to the patient by an adjustable strap 108 having a releasable connector 110, such as a hook and loop fastener. The tube support portion 106 connects the proximal portion of the tube 112, 212 to the anchor 104, and acts to prevent longitudinal movement or slippage of the ETT 100, 200, 300 relative to the patient. The tube holder 102 also substantially maintains the proximal portion 112, 212 of the ETT 100, 200, 300 at a selected position relative to the patient's mouth. In the embodiment shown, the tube support 106 is an arcuately shaped arm that includes a connector 116 at one end that is adapted for releasable attachment to the proximal portion 112 of the ETT 100. Additional details of the tube holder 102 of FIG. 1 are described below.

As shown in FIG. 1, the apparatus 10 also may include one or more tube location devices or tube locators 146, 148 on a distal portion 121, 221 of the ETT 100, 200, 300. The tube location devices 146, 148 are configured to permit medical personnel to readily determine the position of the distal portion 121, 221 of the ETT 100, 200, 300 in a patient's airway without disturbing the patient or the ETT 100, 200, 300. Further details of the tube location devices 146, 148 are described below.

One embodiment of an ETT 100 according to the invention is shown in FIGS. 2A and 2B. As shown in FIG. 2A, the ETT 100 can include an extensible and contractible portion 120 between the proximal end 113 and the distal end 119 of the tube, and an expandable cuff 118, 218 on the distal portion 121 of the tube 100. In FIG. 2A, the extensible and contractible portion 120 is shown in a substantially fully compressed state. When the extensible and contractible portion 120 is fully compressed, the ETT 100 is at its shortest overall length, and the proximal end 113 is nearest the cuff 118. In FIG. 2B, the extensible and contractible portion 120 is shown in a fully extended state. When the extensible and contractible portion 120 is fully extended, the ETT 100 is at its longest overall length, and the proximal end 113 is farthest from the cuff 118. In the embodiment shown in FIGS. 2A and 2B, the extensible and contractible portion 120 is integrally formed with the proximal tube portion 112 and the distal tube portion 121 of the ETT 100. In this embodiment, the extensible and contractible portion 120 may be formed by a series of cooperating folds or corrugations 123 in the wall of the tube. The accordion-like corrugations 123 permit the extensible and contractible portion 120 to extend in length as the proximal end 113 of the ETT 100 is urged away from the cuff 118, and to contract in length as the proximal end 113 of the ETT 100 is urged toward the cuff 118. Preferably, the proximal and distal tube portions 112, 121 and dynamic buffer 120 of the ETT 100 are constructed of a suitable medical grade plastic, such as medical grade polyvinyl chloride (PVC), or the like. Other suitable materials also may be used to form the tube 100. Preferably, the proximal and distal tube portions 112, 121 and dynamic buffer 120 of the ETT 100 are substantially transparent or translucent to permit viewing of sputum, foreign bodies, obstructions, blood, or the like as such secretions are suctioned from an intubated patient through the ETT 100.

The ability of the extensible and contractible portion 120 to extend and contract in length provides a dynamic buffer between the distal portion 121 and the proximal portion 112 of the ETT 100. This dynamic buffering effect minimizes the reaction by the distal portion 121 of the ETT 100 and the cuff 118 in response to a displacement or disturbance of the proximal portion 112 in an intubated patient. Preferably, the extensible and contractible portion 120 is sufficiently flexible that the ETT 100 is capable of substantially freely extending and contracting in length when subjected to conditions typically experienced in an intubated patient. In one embodiment, the dynamic buffer 120 is about 8 cm to about 28 cm from the distal end 119 of the ETT 100. Preferably, the dynamic buffer is about 14 cm to about 22 cm from the distal end 119. In one embodiment, the dynamic buffer 120 is positioned on the ETT 100 such that the dynamic buffer 120 is positioned within the confines of an intubated patient's mouth. The dynamic buffer 120 can be about 0.5 cm to about 3 cm in length in a fully contracted state, and preferably is about 1 cm in length when fully contracted. The dynamic buffer can be configured to elongate by about 1 cm to about 5 cm, and preferably is capable of extending about 3 cm. As shown in FIGS. 2A and 2B, the extensible and contractible portion 120 can be slightly greater in diameter than the proximal and distal tube portions 112, 121. For example, the dynamic buffer 120 can be up to about 5 mm larger in diameter. Preferably, the extensible and contractible portion 120 has an inner diameter that is substantially the same as the inner diameters of the proximal and distal tube portions 112, 121, such that the flow of air or other gasses through the ETT 100 is unaffected by the dynamic buffer 120.

The ETT 100 preferably is capable of supplying air or other gasses at a positive pressure of about 100 mm H₂O, and withstanding negative pressures during patient inhalation or suctioning as low as about −35 mm H₂O. The dynamic buffer 120 preferably is sufficiently rigid to resist dilation under expected positive internal pressures, and to resist collapse during expected negative internal pressures.

As shown in FIGS. 2A and 2B, the ETT 100 may include an expandable cuff 118, 218. In the embodiment shown in FIGS. 2A and 2B, the cuff 118, 218 is an inflatable balloon cuff. In FIG. 2A, the balloon cuff 118, 218 is shown in a deflated state. When deflated, the balloon cuff 118, 218 substantially conforms to the outer surface of the distal portion 121 of the ETT 100. The balloon cuff 118, 218 includes an inflatable bladder adapted to be inflated about the distal tube portion 121 such that when inflated, the cuff 118, 218 seals the region between the distal tube portion 121 and an intubated patient's airway. The balloon cuff 118, 218 may be inflated by air introduced through an inflation tube 144. The cuff 118, 218 also may be deflated by permitting air to pass from the cuff 118, 218 through the inflation tube 144. Preferably, the inflation tube 144 is as thin as possible, such that the inflation tube 144 does not substantially interfere with insertion of the ETT 100 into a patient's airway.

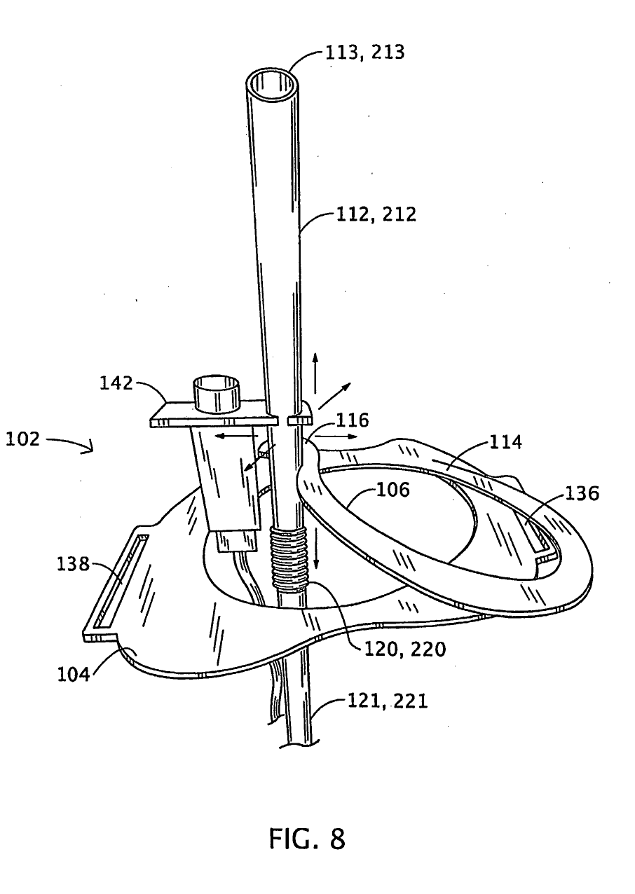

As shown in FIGS. 2A and 2B, air may be supplied to the inflation tube 144 and balloon cuff 118, 218 by an inflation device 142. In the embodiment shown, the inflation device 142 is a manually operable syringe pump of a type known in the art. As shown in FIG. 8, the inflation device 142 preferably is positioned on an ETT 100, 200, 300 such that an operator can activate the inflation device and insert the ETT 100, 200, 300 with one hand, while manipulating a laryngoscope with the other hand. The balloon cuff 118, 218 is shown in an inflated state in FIG. 2B. Further details of the cuff 118, 218 are described below.

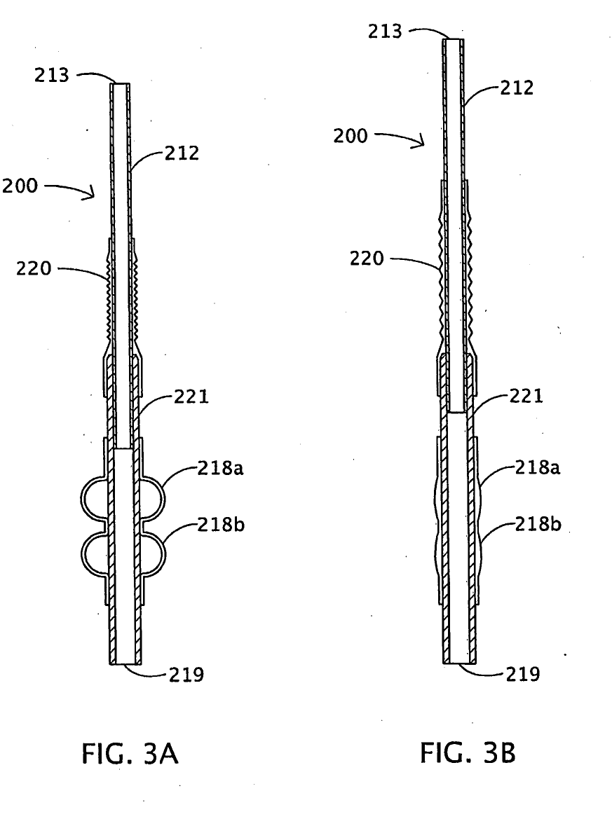

A second embodiment 200 of an ETT for use in an endotracheal intubation apparatus according to the invention is shown in FIGS. 3A and 3B. In this embodiment, the ETT 200 includes proximal and distal tube portions 212, 221. As shown in FIGS. 3A and 3B, the proximal tube portion 212 may have a smaller diameter than the distal tube portion 221, and be slidingly engaged in a bore of the distal tube portion 221. Alternatively, the distal tube portion 221 may have a smaller diameter than the proximal tube portion 112, and be slidingly engaged in a bore of the proximal tube portion 112. This arrangement permits telescopic movement between the proximal and distal tube portions 212, 221. In the contracted state shown in FIG. 3A, the proximal tube portion 212 is inserted into the distal tube portion 221 such that the proximal end 213 of the ETT. is at its closest point to cuffs 218a, 218b. In the extended state shown in FIG. 3B, the proximal tube portion 212 is partially withdrawn from the distal tube portion 221 such that the proximal end 213 of the ETT 200 is at its farthest point from the cuffs 218a, 218b. In one embodiment, the total range of axial movement of the proximal tube portion to the distal tube portion 221 and cuff 218 is between about 1 cm and about 5 cm. The ETT 200 also may have larger or smaller ranges of relative movement. As shown in FIGS. 3A and 3B, the ETT 200 may be provided with an extensible and contractible sleeve 220 that envelopes the telescoping interface between the proximal and distal tube portions 212, 221. The sleeve 220 can be bonded to the tube portions 212, 221 to form a substantially airtight seal between the tube portions 212, 221, such that air introduced to a patient through the ETT 200 cannot escape and cannot bypass the patient's lungs.

As shown in FIGS. 3A and 3B, the extensible and contractible sleeve 220 may include a series of folds or corrugations 223 that cooperate to permit the sleeve 220 to extend and contract in length as the tube sections 212, 221 slide relative to each other. Preferably, the tube portions 212, 221 and sleeve 220 of the ETT 200 are constructed of one or more suitable medical grade plastics, such as medical grade polyvinyl chloride (PVC). Other suitable materials also may be used to form the tube components 212, 221, 220. Preferably, the proximal and distal tube portions 212, 221 and sleeve 220 of the ETT 200 are substantially transparent or translucent to permit viewing of sputum, foreign bodies, obstructions, blood, or the like as such secretions are suctioned from an intubated patient through the ETT 200.

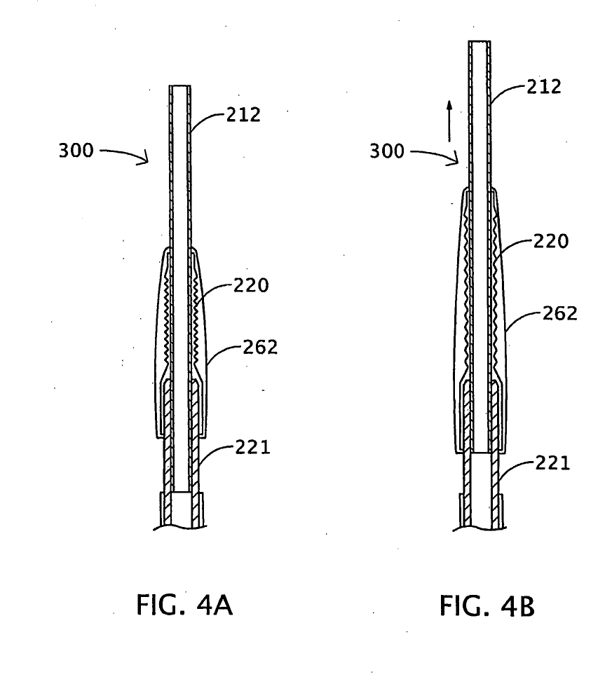

In one embodiment, the dynamic buffer 220 is about 8 cm to about 28 cm from the distal end 219 of the ETT 200. Preferably, the dynamic buffer is about 14 cm and about 22 cm from the distal end 219. In one embodiment, the dynamic buffer 220 is positioned on the ETT 100 such that the dynamic buffer 220 is positioned within the confines of an intubated patient's mouth. The dynamic buffer 220 can be about 0.5 cm to about 3 cm in length in a fully contracted state, and preferably is about 1 cm in length. The dynamic buffer 220 also can be configured to elongate by about 1 cm to about 5 cm, and preferably is capable of extending about 3 cm. As shown in FIGS. 4A and 4B, the extensible and contractible portion 220 can be slightly greater in diameter than the proximal and distal tube portions 212, 221. For example, the dynamic buffer 220 can be up to about 5 mm larger in diameter. Like the embodiment 100 described above, the ETT 200 preferably is constructed to withstand internal pressures from about −35 mm H₂O to about +100 mm H₂O without dilation or collapse.

Another embodiment 300 of an ETT according to the invention is shown in FIGS. 4A and 4B. In this embodiment, the tube 300 is substantially the same as the ETT 200 described above, and further includes a protective sheath 262 over the extensible and contractible sleeve 220. The protective sheath 262 can have a smooth outer shape and smooth outer surface that minimizes potential abrasion and irritation of adjacent patient tissue. For example, the protective sheath 262 may shield surrounding patient tissue from abrasion by the corrugations 223 of the extensible and contractible sleeve 220. The protective sheath 262 is constructed of a material that is sufficiently stretchable and flexible to permit the relative telescopic movement between the proximal and distal tube portions 212, 221. For example, the protective sheath 262 may be constructed of a thin medical grade natural or synthetic rubber, or the like. A similar protective sheath 262 can be placed around the extensible and contractible portion 120 of the one-piece tube construction shown in FIGS. 2A and 2B.

As shown in FIGS. 1, 2A and 2B, the ETT 100, 200, 300 can include one or more tube location devices 146, 148. In the embodiments shown, the tube location devices 146, 148 are in the form of rings affixed about the distal tube portion 121 of the ETT 100, proximate to the proximal end of the cuff 118, 170, 218. The tube location devices 146, 148 may be any device that permits medical personnel to determine the position of the distal portion 121, 221 and cuff 118, 218 of the ETT 100, 200, 300 in an intubated patient without disturbing the ETT 100, 200, 300. For example, tube location device 146 may be a fiber optic lamp that is capable of emitting sufficient light to be visible through the trachea and skin of a patient in the region between an intubated patient's sternal notch and cricoid cartilage. Light may be supplied to the fiber optic lamp 146 from a suitable light source via a fiber optic light conductor 147, or the like. By visibly observing and marking the location of the light-emitting location device 146 in a patient after the patient has been properly intubated with the ETT 100, 200, 300, medical personnel can readily determine any change in position of the ETT 100, 200, 300 by determining whether any associated visible change in the position of the location device 146 in the patient has occurred.

In another embodiment, tube location device 148 may include a ring of ferrous or magnetic material. In this embodiment, the position of the tube location device 148 (and thus the position of the ETT 100, 200, 300) can be ascertained using an x-ray apparatus or magnetometer. By visibly observing and marking the location of the location device 148 in a patient after the patient has been properly intubated with the ETT 100, 200, 300, medical personnel can readily determine any change in position of the ETT 100, 200, 300 by observing any associated change in the position of the location device 146 in the patient.

As shown in FIG. 1, an intubation apparatus 10 according to the invention can include an ETT 100, 200, 300 having an expandable cuff 118, 170, 218. In the embodiment shown in FIGS. 3A and 3B, the ETT 200 includes a pair of balloon cuffs 218a, 218b. In this embodiment, the cuffs 218a, 218b have a rounded or ovoid shape similar to conventional balloon cuffs. The ETT 100, 200, 300 may include one or more balloon cuffs 218a, 218b. Multiple balloon cuffs 218a, 218b act to distribute pressure on surrounding tissues over a greater surface area, thereby minimizing the potential for harm to the surrounding tissue of an intubated patient. Preferably, the balloon cuffs 218a, 218b are substantially longer than traditional balloon cuffs to further distribute pressure loadings on the surrounding tissue. For example, whereas traditional balloon cuffs are about 33 mm in length, the balloon cuffs 218a, 218b preferably are about 33-40 mm in length.

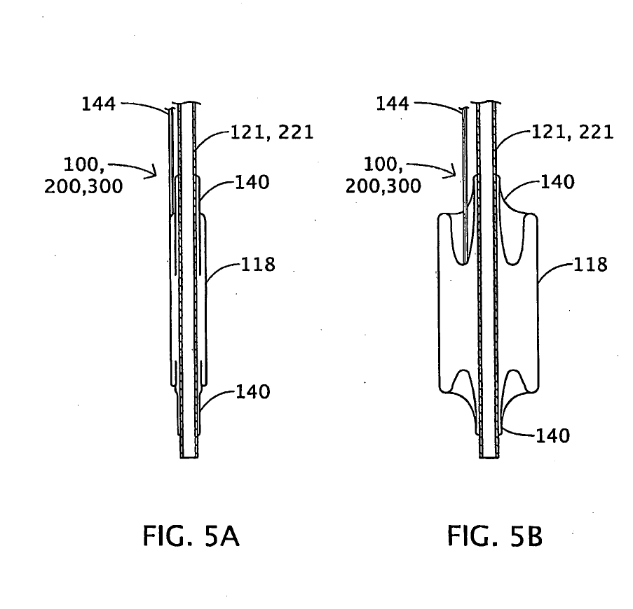

Another embodiment of an expandable balloon cuff 118 for use in the invention is shown in FIGS. 5A and 5B. In this embodiment, the balloon cuff 118 has a substantially cylindrically shaped outer surface 124. Preferably, the cuff 118 is constructed of an elastically stretchable material, such as medical grade rubber or plastic, or the like. When the balloon cuff 118 is in a deflated, unexpanded state as shown in FIG. 5A, the outer surface 124 preferably substantially conforms to the outer wall of the distal tube portion 121, 221 of the ETT 100, 200, 300. Thus, when deflated, the cuff 118 will not interfere with insertion of the distal tube portion 121, 221 into a patient's airway. The cuff 118 may be deflated by applying a vacuum via an inflation tube 144, and/or by manually compressing the cuff 118. When air is introduced to the balloon cuff 118 through the inflation tube 144, the shape of the cuff 118 causes the outer surface 124 to radially expand, such that the outer surface 124 of the cuff 118 maintains a substantially cylindrical shape. When the cuff 118 is expanded in an intubated patient's airway, the cuff 118 forms a substantially airtight seal between the distal tube portion 121, 221, and the inner wall of the patient's trachea. Preferably, the cylindrical cuff 118 has a longitudinal length of about 30-40 mm, compared to lengths of about 33 mm for conventional ovoid balloon cuffs. The cylindrical outer surface 124 and longer length of the cuff 118 provides a greater surface area for contacting surrounding tissue of an intubated patient than conventional rounded or ovoid balloon cuffs. Accordingly, pressure loads between the expanded cuff 118 and the patient's surrounding tissue are distributed over a greater surface area than occurs with conventional cuffs. Thus, the contact pressure between the cuff 118 and surrounding patient tissue is substantially less than corresponding contact pressures produced by conventional cuffs, thereby reducing the potential for pressure necrosis or vascular compromise in an intubated patient. The outer surface 124 of the cuff 118 may be dimpled or otherwise textured to enhance the ability of the cuff to grip surrounding patient tissue. For most patients, the trachea generally narrows in a distal direction. This general narrowing of the trachea acts to at least partially limit distal migration of an ETT in such patients. Distal migration of an ETT 100, 200, 300 according to the invention is further limited by the cylindrically shaped balloon cuff 118, and by the responsive extension and contraction of the dynamic buffer 120, 220 described above.

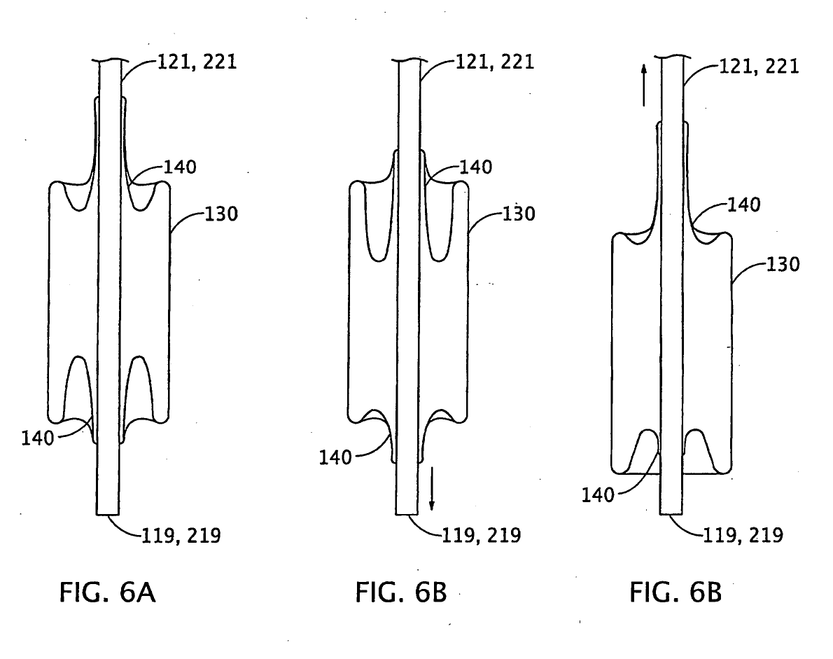

As shown in FIGS. 5A and 5B, the outer portion 130 of the inflatable balloon cuff 118 is attached to the distal tube portion 121, 221 by connecting material 140. Preferably, connecting material 140 is an elastically stretchable and pliable membrane constructed of medical grade rubber or plastic, or the like. As shown in FIGS. 6A-6C, the connecting material 140 permits at least some relative axial movement between the distal tube portion 121, 221 and the outer portion 130 of the cuff 118. The relative movement between the cuff 118 and tube 121, 221 can be, for example, in response to movement of an intubated patient's head or neck. The connecting material 140 also permits the cuff 118 and tube 121, 221 to return their initial relative positions when the patient's head or neck return to their initial positions. The connecting material 140 may be integrally formed with the cuff 118.

FIG. 6A shows an outer portion 130 of the cuff 118 in a central position on the distal tube portion 121, 221 of an ETT 100, 200, 300. In this position, the connecting material 140 is in a substantially relaxed or static state. As shown in FIG. 6B, the stretchable and pliable connecting material 140 permits the outer portion distal tube portion 121, 221 to move at least some axial distance in a distal direction. In other words, the connecting material 140 permits the distal end 119, 219 of the distal tube portion 121, 221 to displace in a distal direction in an intubated patient, though the outer portion 130 of the cuff 118 remains at a substantially fixed position in the patient's trachea. Conversely, as shown in FIG. 6C, the stretchable and pliable connecting material 140 permits the outer portion distal tube portion 121, 221 to move at least some axial distance in a proximal direction in an intubated patient. In other words, the connecting material 140 permits the distal end 119, 219 of the distal tube portion 121, 221 to move in a proximal direction in an intubated patient, though the outer portion 130 of the cuff 118 remains at a substantially fixed position in the patient's trachea. Therefore, the flexible connecting material 140 of the cuff 118 acts as a resilient buffer that minmizes the potential for displacement of the outer portion 130 of the cuff 118 in an intubated patient in response to distal and/or proximal movement of the distal tube portion 121, 221 caused by a patient's bodily movement, or by disturbance of the ETT 100, 200, 300 by medical personnel.

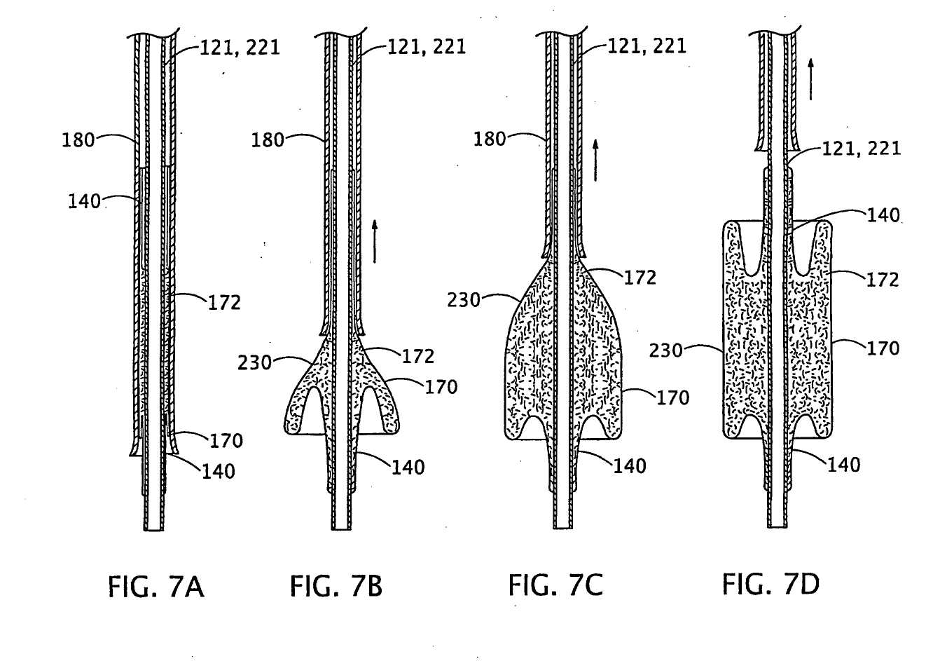

Another embodiment of an expandable balloon cuff 170 for use in an ETT 100, 200, 300 according to the invention is shown in FIGS. 7A-7D. In this embodiment, the cuff 170 includes a resilient compressible material 172. The resilient material 172 may be enclosed by a resilient cover 230. In this embodiment, no inflation device is required to inflate the cuff 170. Preferably, like the balloon cuff 118 described above, the cuff 170 has a substantially cylindrical outer shape in an expanded state, as shown in FIG. 7D. The cylindrical outer shape of the cuff 218 provides a larger surface area for contacting surrounding tissue of an intubated patient than conventional rounded or ovoid balloon cuffs. The resilient compressible material 172 may be a sponge rubber, or a foamed plastic material, such as foamed polyurethane, or the like. The resilient compressible material 172 biases the cuff 170 toward a fully expanded state, like that shown in FIG. 7D. Accordingly, before insertion of the ETT 100, 200, 300 into a patient's trachea, the cuff 170 should be compressed.

As shown in FIGS. 7A-7C, the cuff 170 may be compressed and held in a compressed state by a removable sheath 180. As shown in FIG. 7A, when the sheath 180 is placed around the expandable cuff 170, the cuff 170 is compressed and constrained about the outer diameter of the distal tube portion 121, 221. Accordingly, the sheath 180 effectively reduces the outer diameter of the cuff 170, and thereby facilitates insertion of the distal tube portion 121, 221 and cuff 170 into a patient's trachea. As shown in FIGS. 7B and 7C, once the distal tube portion 121, 221 and cuff 170 are at a selected position within a patient's trachea, the removable sheath 180 can be withdrawn in a proximal direction from the ETT 100, 200, 300, thereby permitting the resilient compressible material 172 and cuff 170 to gradually expand within the patient's trachea, such as in about 30 seconds. Once thus fully expanded, the cuff 170 provides a substantially airtight seal between the distal tube portion 121, 221 and the surrounding tissue of the patient. The embodiment shown in FIGS. 7A-7D is particularly well suited for use in military combat situations.

Alternatively, the resilient compressible material 172 my be compressed around the distal tube portion 121, 221 by extracting entrapped air from the material by applying a vacuum through a small diameter tube, or the like. In another alternative, the resilient compressible material 172 may be initially compressed by manually squeezing the cuff 170, and then permitting the cuff 170 to gradually expand after insertion into a patient's airway.

Like the balloon cuff 118 described above, the self-expanding balloon cuff 170 preferably is attached to the distal tube portion by an elastically stretchable and pliable connecting material 140. Once the distal tube portion 121, 221 and cuff 170 are properly positioned within a patient's airway, the connecting material 140 permits at least some proximal and distal movement of the distal tube portion 121, 221 relative to the cuff 170, similar to the relative displacement illustrated in FIGS. 6A-6C for balloon cuff 118. An outer surface of the cuff 170 may include dimples or other textured features to minimize the potential for slippage or migration of the expanded cuff 170 within a patient's trachea.

As shown in FIGS. 1 and 8, the invention also includes an ETT holder 102. As described above, the tube holder 102 is configured to substantially maintain an ETT 100, 200, 300 in a preferred position in an intubated patient. For example, the tube holder 102 may be configured to substantially maintain the proximal end 113 and proximal portion 112 of an ETT 100 at a central or other oral position of the patient, and to minimize movement or slippage of the ETT 100 in an intubated patient once the ETT 100 has been properly positioned in the patient. In the embodiment shown in FIG. 8, the tube holder 102 includes an anchor 104 and a tube support 106. The anchor 104 is secured to the patient proximate to the patient's mouth. Preferably, the anchor 104 is shaped to substantially conform to the contours of a patient's face, and may be configured to surround the patient's mouth. As shown in FIG. 1, the anchor 104 can be secured to a patient by an adjustable strap 108 having a releasable connector 110, such as a hook and loop fastener. The anchor 104 may include a pair of opposed slots 136, 138 for attaching ends of the strap 108 to the anchor 104. Preferably, the anchor 104 has a smooth outer surface to maximize patient comfort.

The tube support portion 106 connects a proximal tube portion 112 of an ETT 100 to the anchor 104, and acts to resist movement and minimize slippage of the ETT 100 relative to the patient. The tube support 106 also substantially maintains the proximal portion 112 of the ETT 100 at a selected position relative to the patient's mouth. In the embodiment shown, the tube support 106 is an arcuately shaped arm that includes a connector 116 at one end that is adapted for releasable attachment to the proximal portion 112 of the ETT 100. In the embodiment shown, the connector 116 includes a U-shaped hooked portion that is configured to securely and releasably grip the outer diameter of the ETT 100. Alternatively, the connector 116 may be a bracket, a screw, a clamp, or any other form capable of releasably grasping the proximal tube portion 112, 212. Preferably, the tube support 106 is sufficiently rigid to bias the tube 100 toward a selected position relative to a patient, and also is sufficiently flexible to permit at least some resilient movement of the connector 116 and an attached proximal tube portion 112, 212 in substantially any direction, as indicated by the arrows in FIG. 8. In other words, the tube support 106 is sufficiently flexible to permit the connector 116 and an attached proximal tube portion 112, 212 to be displaced distally, proximally, or laterally relative to the anchor 104. Accordingly, the proximal portion 112 of an attached ETT 100 can be selectively and temporarily repositioned by medical personal to gain access to a patient's mouth, for example, while the tube support 106 acts to at least partially restrict the amount of displacement of the attached proximal tube portion 112. In addition, the flexibility of the tube support 106 accommodates movements of the head and neck of an intubated patient, while also permitting a cuff 118, 218 on a distal portion of the attached ETT 100, 200, 300 to remain substantially unaffected at a fixed position within a patient's trachea, thus minimizing the possibility of tracheal mucosal damage resulting from rubbing by the cuff 118, 218.

Preferably, the anchor 104 and tube support 106 are constructed of a single piece of material. For example, the anchor and tube support 106 may be molded together from a medical grade plastic, or the like. Alternatively, the tube support 106 and anchor 104 may be constructed separately. When constructed separately, the fixed end 114 of the tube support 106 can be welded or glued to the anchor 104, fastened with one or more suitable fasteners, or otherwise connected to the anchor 104.

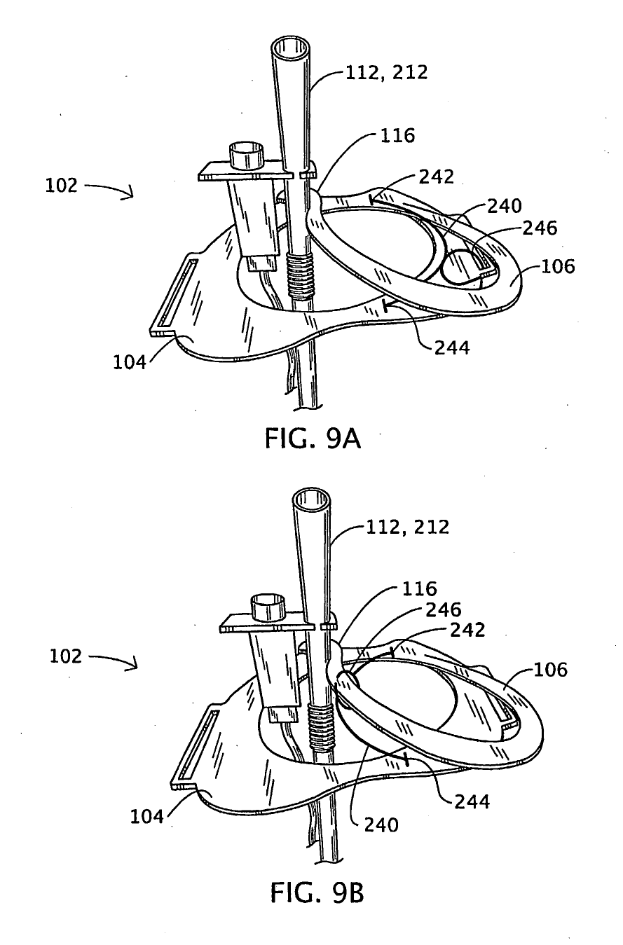

As shown in FIGS. 9A-9B, the tube holder 102 also may include a locking member 240. The locking member 240 releasably fixes the position of the tube support 106 relative to the anchor 104. As shown in FIG. 9A, the locking member 240 may be an arch-shaped element pivotally attached to the anchor at hinges 242, 244. The hinges 242, 244 permit the locking member 240 to pivot between an unlocked position (shown in FIG. 9A) and a fully locked position (shown in FIG. 9B). The locking member 240 may include a gripping portion 246. The gripping portion 246 is configured to releasably engage the tube support arm 106 when the locking member is in a locked position. Accordingly, when the locking member is selectively pivoted to the fully locked position (shown in FIG. 9B) and the tube support 106 is engaged with the gripping portion 246, the tube support arm 106 is substantially restrained against movement relative to the anchor 104. Locking the tube holder 102 in this way may be desirable during procedures that may pull or push the ETT 100, 200, 300, and may cause unwanted displacement of the ETT 100, 200, 300 from its selected position in a patient's trachea. For example, use of the locking device 240 may be desirable when a bag-valve-mask (BVM) is being used to introduce air through the ETT 100, 200, 300. Once the potentially disruptive procedure is complete, and the patient is returned to a secure and stable condition (such as being placed on a ventilator), the locking member 240 can be released from the tube support 106. Once the locking member 240 is disengaged from the tube support 106, the support arm 106 is again able to resiliently respond to patient movement, and to help maintain the position of the ETT 100, 200, 300 in the intubated patient during such movement.

The above description of various embodiments of the invention is intended to describe various aspects of the invention, and not to limit the invention thereto. A person of ordinary skill in the art will understand that various modifications can be made to the specifically described embodiments without departing from the scope of the invention. All such modifications are intended to be within the scope of the appended claims.

1. An endotracheal intubation apparatus comprising:
(a) a tube comprising a distal end, a proximal end, and at least one extensible and contractible portion;
(b) at least one cuff on the tube proximate to the distal end, the cuff being movably attached to the tube such that the cuff is capable of at least some movement in a proximal direction and in a distal direction relative to the tube;
(c) a tube holder comprising a resilient support configured to substantially bias the proximal end of the tube toward a selected oral position in a patient; and
(d) at least one tube locator that is adapted to indicate the position of a distal portion of the tube within the patient.

2. An endotracheal intubation apparatus according to claim 1, wherein the extensible and contractible portion comprises at least one corrugated tube section.

3. An endotracheal intubation apparatus according to claim 1, wherein the tube comprises a distal tube portion and a proximal tube portion having ends that are telescopically engaged to form the extensible and contractible portion.

4. An endotracheal intubation apparatus according to claim 3, and further comprising a sleeve over the extensible and contractible portion.

5. An endotracheal intubation apparatus according to claim 4 wherein the sleeve is at least partially corrugated.

6. An endotracheal intubation apparatus according to claim 1, wherein the cuff comprises an inflatable balloon cuff.

7. An endotracheal intubation apparatus according to claim 1, wherein the cuff comprises a compressible resilient material.

8. An endotracheal intubation apparatus according to claim 1, wherein the cuff is movably attached to the tube by at least one flexible connector.

9. An endotracheal intubation apparatus according to claim 1, wherein the cuff has a substantially cylindrical outer shape in an expanded state.

10. An endotracheal intubation apparatus according to claim 1, wherein the tube holder comprises an anchor that is removably mountable on the patient proximate to the patient's mouth, and wherein the resilient support comprises a resilient arm having a first end connected to the anchor, and a second end configured to removably receive the proximal end of the tube.

11. An endotracheal intubation apparatus according to claim 10 wherein the resilient arm permits at least some movement of the second end and the proximal end of the tube received therein in substantially any direction.

12. An endotracheal intubation apparatus according to claim 11 wherein the resilient arm has an arcuate shape.

13. An endotracheal intubation apparatus according to claim 11, and further comprising an adjustable strap for removably mounting the anchor on the patient.

14. An endotracheal intubation apparatus according to claim 1 wherein the tube locator comprises a light-emitting member.

15. An endotracheal intubation apparatus according to claim 1 wherein the tube location device comprises a ferrous member.

16. An endotracheal intubation apparatus according to claim 1 wherein the tube locator comprises a magnetic member.

17-18. (canceled)

19. An endotracheal tube comprising a distal end, a proximal end, and an extensible and contractible portion therebetween, wherein the tube comprises a distal tube portion and a proximal tube portion that are telescopically interconnected to form the extensible and contractible portion.

20. An endotracheal tube according to claim 19, and further comprising a sleeve over the extensible and contractible portion.

21. An endotracheal tube according to claim 20 wherein the sleeve includes a plurality of corrugations.

22. An endotracheal tube comprising a distal end and at least one cuff on the tube proximate to the distal end, the cuff being movably attached to the tube such that the cuff is capable of at least some movement in a proximal or distal direction on the tube.

23. An endotracheal tube according to claim 22, wherein the cuff comprises an inflatable balloon cuff.

24. An endotracheal tube according to claim 22, wherein the cuff comprises a resilient compressible material.

25. An endotracheal tube according to claim 22, wherein the cuff is movably attached to the tube by at least one flexible member.

26. An endotracheal tube according to claim 22, wherein the cuff has a substantially cylindrical outer shape when in an unrestrained expanded state.

27. A tube holder for an endotracheal tube comprising a resilient support configured to substantially bias the proximal end of the tube toward a central oral position of a patient.

28. A tube holder according to claim 27, wherein the tube holder comprises an anchor that is removably mountable on the patient proximate to the patient's mouth, and wherein the resilient support comprises a resilient arm having a first end connected to the anchor, and a second end configured to removably receive the proximal end of the tube.

29. A tube holder according to claim 28 wherein the resilient arm permits at least some movement of the second end and the proximal end of the tube received therein in substantially any direction.

30. A tube holder according to claim 28, and further comprising an adjustable strap for removably mounting the anchor on the patient.

31. An endotracheal tube comprising a tube locator on the tube proximate to a distal end of the tube, the tube locator being adapted to indicate the location of the distal end of the tube in an intubated patient.

32. An endotracheal tube according to claim 31 wherein the tube locator comprises at least one light-emitting member.

33. An endotracheal tube according to claim 32 wherein the light-emitting member comprises at least one fiber optic element.

34. An endotracheal tube according to claim 31 wherein the tube locator comprises at least one magnetic member.

35. An endotracheal tube according to claim 31 wherein the tube locator comprises at least one ferrous member.
